(19) United States
(12) Patent Application Publication
(10) Pub. No.: US 2022/0143363 A1
(43) Pub. Date: May 12, 2022

(54) ABSORBENT DEVICE FOR USE WITH CATHETER

(71) Applicant: CIC FUND SECURITISATION S.A., Luxembourg (LU)

(72) Inventors: Matthew G. MCINTYRE, Theodore, AL (US); Peter T. FALKNER, Mobile, AL (US); Deneen T. PLESSALA, Mobile, AL (US); Michael HADDAD, Atlanta, GA (US); Kirby J. PLESSALA, Mobile, AL (US)

(21) Appl. No.: 17/604,666

(22) PCT Filed: Apr. 17, 2020
(86) PCT No.: PCT/US2020/028753

§ 371 Date: Oct. 18, 2021

Related U.S. Application Data
(60) Provisional application No. 62/835,025, filed on Apr. 17, 2019.

(51) Int. Cl. A61M 25/00

(52) U.S. Cl. CPC A61M 25/0082; A61M 2205/02; A61M 2205/0227; A61M 2205/584

(57) ABSTRACT

An absorbent cuff for use with a male patient wearing a catheter is provided, the cuff including a nonpermeable body operable to receive and enclose an absorbent material and to prevent leakage of a fluid retained. A circumferentially concave opening is operable for positioning on the catheter below the head of a patient's penis and surround the point of insertion of the catheter into the patient's urethra. A catheter passage is operable to enclose a catheter and to receive and retain fluid discharged from a catheter. A slit enables a catheter to be inserted into the catheter passage of the absorbent material. An absorbent pad operable for use with female patients includes an absorbent material, a nonpermeable backing, a catheter passage, and a slit to allow for insertion and removal of a catheter.

### CROSS REFERENCE TO RELATED APPLICATIONS

This application claims priority to U.S. Provisional Patent Application No. 62/835,025, filed Apr. 17, 2019, the entirety of which is incorporated by reference as if fully disclosed herein.

### BACKGROUND

The present invention relates to a device for absorbing bodily fluids, and more particularly to a device for absorbing excess fluids discharged by a urethral irrigation catheter.

Urethral catheters are devices well known in the art and are used to drain urine from the bladder. When a urinary catheter is inserted into the bladder, germs can migrate along the catheter and cause infection in the bladder or kidney; resulting in a catheter-associated urinary tract infection (or “CAUTI”). CAUTIs are the most common of hospital-acquired infections.

The physiology of the urethra contains natural defenses to these infections. Primarily, the act of urination flushes everything, including bacteria that may collect in and around the urethra. Further, there are glands in the urethra that secrete protecting mucus. However, when typical urethral catheters are inserted into the system, these defenses are not activated; there is no act of “flushing” outside the catheter. Recent developments in the art to solve this problem include the development of irrigation catheters which essentially include a separate lumen in addition to the drainage lumen that circulates sterile fluid (e.g. normal saline, antiseptic, antimicrobial, or antibiotic) from the catheter, into the urethra, and allow the fluid to drain out of the body. An example of such an irrigation catheter is disclosed in International Application No. PCT/US2017/026450, the contents of which are fully incorporated into this application. This irrigation process flushes bacteria out of the body, thereby preventing infection. Unfortunately, an inevitable problem with such an irrigation system is that the irrigation fluid will drain out of the urethra and all over the patient, unless there is a device to catch and absorb the excess fluid.

### BRIEF SUMMARY OF INVENTION

The present invention seeks to create devices that attach comfortably to the catheter below the meatus of a male or a female patient to absorb excess fluid, such as irrigation fluid, while not impeding the function of the catheter. In accordance with embodiments of the invention, an absorbent cuff for use with a male patient wearing a catheter is provided. The absorbent cuff includes a nonpermeable body having a distal end opposite a proximal end. The body defines an interior operable to receive and enclose an absorbent material. The body is further operable to prevent leakage of a fluid retained within an absorbent material contained within the body. A circumferentially concave opening is disposed at the proximal end of the body and is operable for positioning on the catheter below the head of a patient's penis and surround the point of insertion of the catheter into the patient's urethra. An absorbent material is disposed within the interior of the body. The absorbent material substantially fills the interior defined by the body and the circumferentially concave opening. The absorbent material defines a circumferentially concave receiving surface disposed within the circumferentially concave opening of the body. The absorbent material defines a catheter passage operable to enclose a catheter. The absorbent material is operable to receive and retain fluid discharged from a catheter. A slit is disposed along a length of the body and absorbent material operable to enable a catheter to be inserted into the catheter passage of the absorbent material.

In accordance with embodiments of the invention, an absorbent cuff for use with a male patient wearing a catheter is provided. The absorbent cuff includes a nonpermeable body having two opposing halves. The body has a distal end opposite a proximal end and defines an interior operable to receive and enclose an absorbent material. The body is operable to prevent leakage of a fluid retained within an absorbent material. A locking mechanism is operable to secure the two opposing halves and operable to create a leakproof seal along the length of the body. An absorbent material is disposed within the interior of the body. The absorbent material defines a catheter passage operable to enclose a catheter. The absorbent material is operable to receive and retain fluid discharged from a catheter. A circumferentially concave opening is disposed at the proximal end of the body and is operable for positioning on the catheter below the head of a patient's penis and to surround the point of insertion of the catheter into the patient's urethra.

In accordance with embodiments of the invention, an absorbent pad for use with a female patient wearing a catheter is provided. The absorbent pad includes an absorbent material operable to receive and retain fluid discharged from a catheter. The absorbent material has a proximal surface opposite a distal surface. A nonpermeable backing is disposed on the distal surface of the absorbent material and is operable to prevent leakage of a fluid retained within the absorbent material. A catheter passage is defined by the absorbent material and the nonpermeable backing. The catheter passage is operable to receive and form seal around the catheter, said passage further operable to prevent the absorbent pad from moving relative to the catheter. A slit extends from the catheter passage to an edge of the absorbent pad and is operable to enable a catheter to be inserted into the catheter passage. A fastener is operable to form a seal across the slit.

In one embodiment, the device comprises a detachable exterior housing having a distal portion and a proximal portion with openings on opposite ends, wherein the distal portion is adapted to hold a material with absorbing properties and the proximal portion is adapted to attach to the catheter below the head of the penis. The absorbing material contained within the distal portion further comprises a channel that allows a catheter to pass through the device while in use. Optionally, two sides of the exterior housing and the contained interior absorbing material can be separated and opened by use of a hinge, or some other attachment means known in the art, in order to allow easier placement of the catheter within the channel inside the device.

An alternative embodiment of the device comprises an exterior non-permeable portion covering an interior permeable portion made from a material with absorbing properties designed to wick liquid away from the penis. Said exterior non-permeable portion and interior permeable portion further comprise a handle portion connected to a head portion with openings on opposite ends and a channel running longitudinally through the center of both portions along the length of the device between the openings, said channel is adapted to accommodate a catheter. Preferably, the surface at the distal end of the head portion is circumferentially concave to create a surface that is adapted to comfortably rest below the head of the penis and surround the point of insertion of the catheter into the urethra. Additionally, there is a crevice extending radially from the channel to the exterior surface of the device and extending longitudinally along the entire length of the device. The crevice allows said portions to be slightly separated in order to allow easy access to the channel for attachment or removal from the catheter. Optionally, in order to create a better seal around the catheter, the device can be sealed using fastening means or a binding device known in the art, including but not limited to for example, the use of hook and loop fasteners, snap or clip fasteners, or a pre-fabricated, semi-flexible holder that clasps around the device.

### BRIEF DESCRIPTION OF THE DRAWINGS

FIG. 1 is a perspective view of an absorbent cuff in accordance with embodiments of the invention.

FIG. 2 is a perspective view of an absorbent cuff in accordance with embodiments of the invention.

FIG. 3 is a perspective view of an absorbent cuff in accordance with embodiments of the invention.

FIG. 4 is a perspective view of an absorbent cuff showing the two sides of the device slightly detached in accordance with embodiments of the invention.

FIG. 5 is a perspective view of an absorbent material in a slightly open position in accordance with embodiments of the invention.

FIG. 6 is a perspective view of an absorbent cuff showing the device in a slightly open position with the absorbing material removed from the exterior housing in accordance with embodiments of the invention.

FIG. 7 is a perspective view of an absorbent material in a slightly open position in accordance with embodiments of the invention.

FIG. 8 is a perspective view of an absorbent cuff showing the device in a slightly open position with the absorbing material removed from the exterior housing in accordance with embodiments of the invention.

FIG. 9 is a front view of an absorbent pad in accordance with embodiments of the invention.

FIG. 10 is a cross sectional view of an absorbent pad in accordance with embodiments of the invention.

### DETAILED DESCRIPTION

The embodiments of the disclosure will be best understood by reference to the drawings, wherein like parts are designated by like numerals throughout. It will be readily understood that the components, as generally described and illustrated in the Figures herein, could be arranged and designed in a wide variety of different configurations or be entirely separate. Thus, the following more detailed description of the embodiments of the system and method of the disclosure, as represented in the Figures is not intended to limit the scope of the disclosure, as claimed, but is merely representative of possible embodiments of the disclosure.

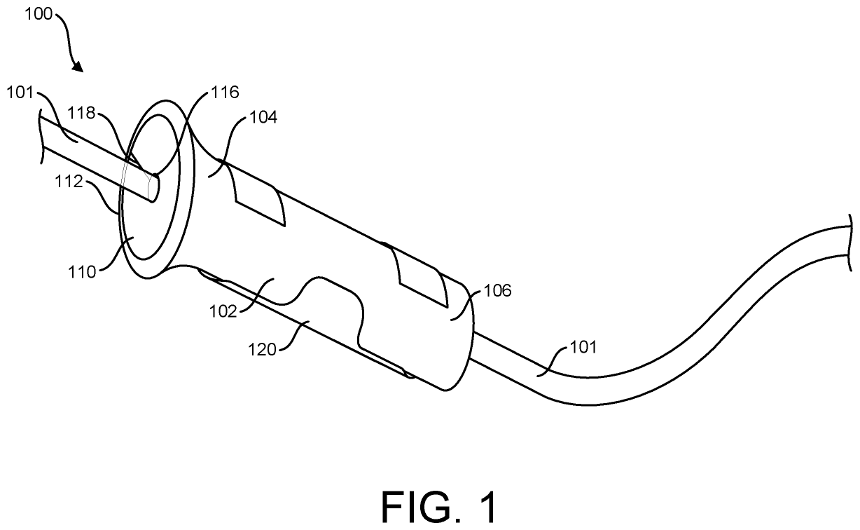

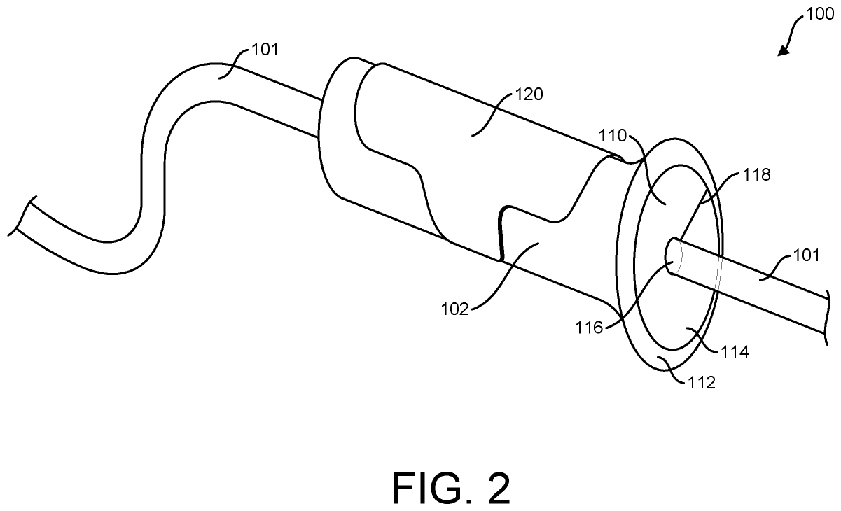

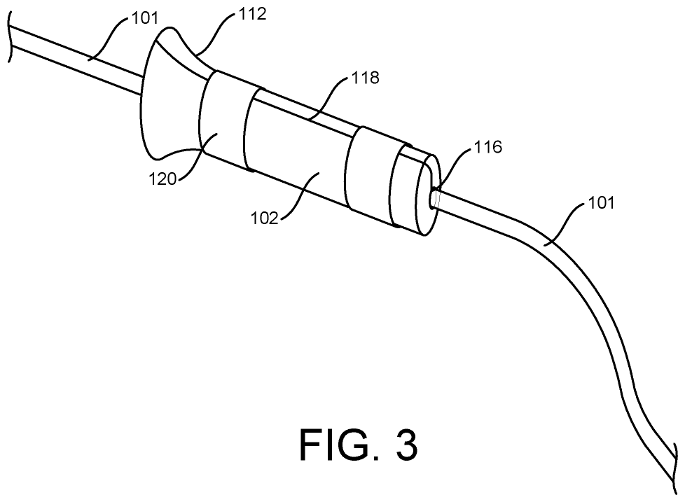

In accordance with embodiments of the invention, as illustrated in FIGS. 1-3, an absorbent cuff 100 for use with a male patient wearing a catheter 101 is provided. The absorbent cuff 100 includes a nonpermeable body 102 having a proximal end 104 opposite a distal end 106. The body 102 may be a molded material, for example. The body 102 defines an interior operable to receive and enclose an absorbent material 110. The body 102 is further operable to prevent leakage of a fluid retained within the absorbent material 110 contained within the body 102. A circumferentially concave opening 112 is disposed at the proximal end 104 of the body 102 and is operable for positioning on the catheter 101 below the head of a patient's penis and surround the point of insertion of the catheter 101 into the patient's urethra. The circumferentially concave opening 112 is configured to create a surface that is adapted to attach comfortably to the catheter 101 below the head of the penis and surround the point of insertion of the catheter 101 into the urethra. The absorbent material 110 is disposed within the interior of the body 102. The absorbent material 110 substantially fills the interior defined by the body 102 and the circumferentially concave opening 112. The absorbent material 110 defines a circumferentially concave receiving surface 114 disposed within the circumferentially concave opening of the body. The absorbent material 110 defines a catheter passage 116 operable to form a seal around and enclose the catheter 101. The absorbent material 110 is operable to receive and retain fluid discharged from the catheter 101, such as from leakage or a flush of the catheter, for example. The absorbent material 110 may be an absorbent material with absorbing properties designed to wick away liquids from the penis, e.g. a super absorbent polymer (“SAP”), or some other comparable material known in the art, such as a material used in a tampon or a diaper, for example. A wicking material is advantageous, as wicking materials rely on capillary action to move fluid within spaces within the absorbent material, which is thereby operable to prevent saturation near the patient. The material used for the absorbent material 110 is adapted to be non-abrasive and non-irritant to the skin of the penis. A slit 118 is disposed along a length of the body 102 and absorbent material 110 operable to enable the catheter 101 to be inserted into the catheter passage 116 of the absorbent material 114. The slit 118 enables the absorbent cuff 100 to be attached to and removed from the catheter 101 without removing the catheter from the patient. This is particularly advantageous as patients may wear catheters for extended periods of time and the absorbent cuff 100 may become full of fluid and need to be replaced prior to the need to remove the catheter 101 from the patient.

In one embodiment, the slit 118 extends radially from the catheter passage 116 to an edge of the absorbent cuff 100 and extends longitudinally along the entire length of the absorbent cuff 100. The portions on either side of the absorbent cuff 100 may be manually separated in order to allow easy access to the catheter passage 116 for attachment or removal from the catheter 118. In order to create a better seal around the catheter 101, for example, the absorbent cuff 100 can be sealed using fastening means known in the art, including for example, hook and loop fasteners, snap or clip button fasteners, or as shown in the figures, a semi-flexible, pre-fabricated holder 120 that clasps around the device.

As illustrated in the embodiment shown in FIGS. 1-3, the thickness of distal end 106 of the absorbent cuff 100 is narrower than the circumferentially concave opening 112 disposed at the proximal end 104. The configuration of the circumferentially concave opening 112 enables a relatively wider surface area to receive fluid and enable wicking and absorption. The relatively narrower distal end 106 enables a lower profile of the device to enable a more comfortable fit for the patient when worn, such as under a hospital gown. The body 102 may be fabricated from a material operable to change color to indicate when the absorbent material 110 is full of fluid. The body 102 may be transparent or include a transparent nonpermeable window to allow a user to see the fluid level contained within the absorbent material 110.

In one embodiment, the absorbent material 110 is manufactured integrally into the nonpermeable body 102. For example, the absorbent material 110 may be affixed to the nonpermeable body 102, dipped into a nonpermeable material operable to harden and coat the absorbent material 110, or a nonpermeable film may be applied to the outer surface of the absorbent material 110.

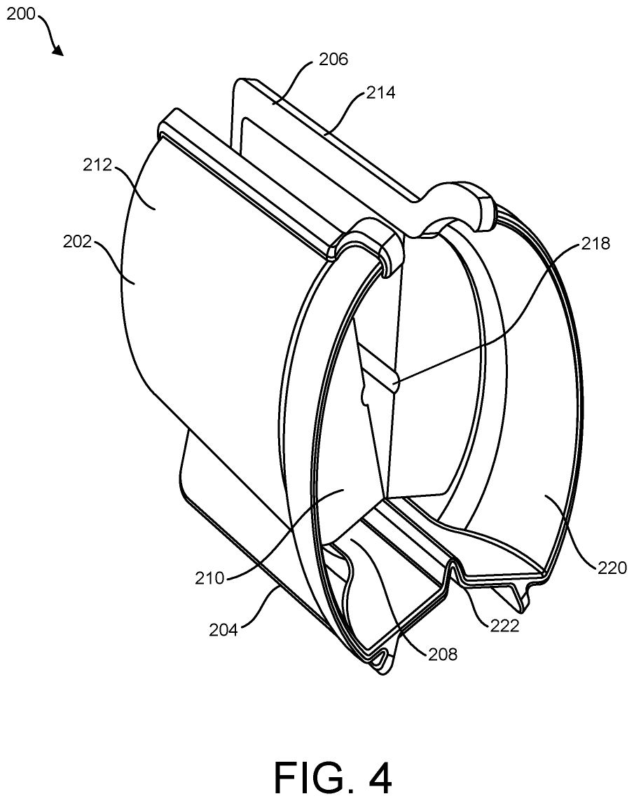

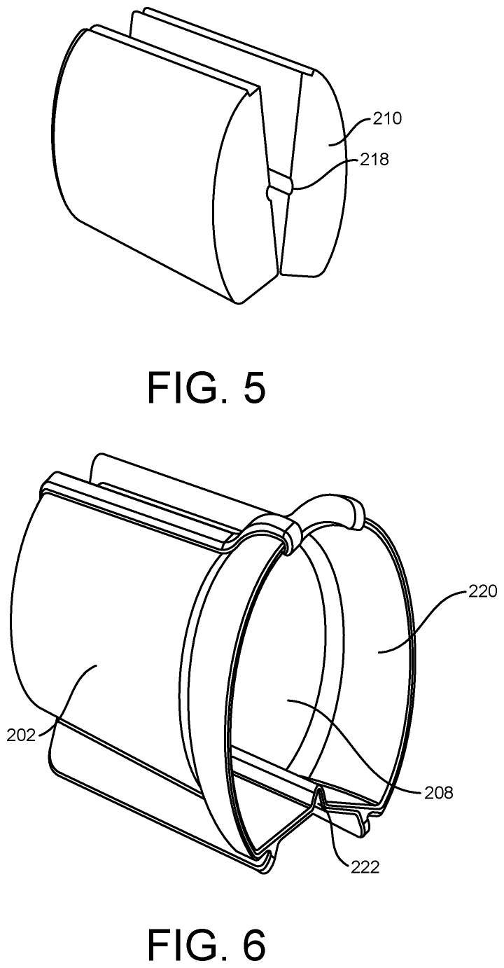

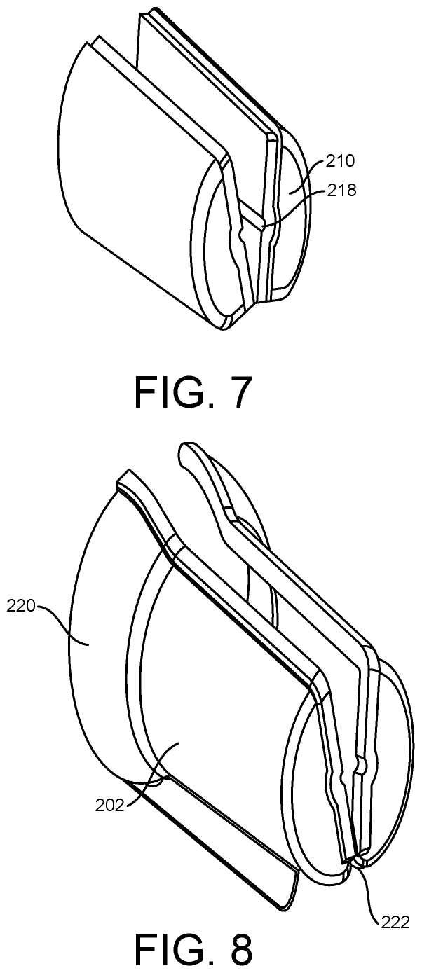

In accordance with embodiments of the invention, as illustrated in FIGS. 4-8, an absorbent cuff 200 for use with a male patient wearing a catheter is provided. The absorbent cuff 200 includes a nonpermeable body 202 having a proximal end 204 opposite a distal end 206 and defines an interior 208 operable to receive and enclose an absorbent material 210. The body 202 has two opposing halves 212 and 214 and may be formed of a molded material, for example. The body 202 is operable to prevent leakage of a fluid retained within the absorbent material 210. A locking mechanism 216 is operable to secure the two opposing halves 212 and 214 and operable to create a leakproof seal along the length of the body 202. An absorbent material 210 is disposed within the interior 208 of the body 202. The absorbent material 210 defines a catheter passage 218 operable to form a seal around and enclose a catheter. The absorbent material 210 is operable to receive and retain fluid discharged from a catheter. A circumferentially concave opening 220 is disposed at the proximal end 204 of the body 202 and is operable for positioning on the catheter 201 below the head of a patient's penis and to surround the point of insertion of the catheter 201 into the patient's urethra. In one exemplary embodiment, the concave opening 220 does not include a portion of the absorbent material 210, creating a reservoir for fluid to accumulate prior to absorption and creating a distance between the patient's penis and the absorbent material 210 to prevent skin irritation.

In order to provide easier placement of the catheter within the absorbent cuff 200, the opposing halves 212 and 214 of the body 202 and the absorbent material 210 contained therein can be opened and separated along a longitudinal hinge 222 running along the length of body 202. Alternatively, the mold of the body 202 can be configured such that the opposing halves 212 and 214 of the body 202 are slightly biased away from each other. Under either scenario, the opposing halves 212 and 214 of the body 202 are secured by attachment means known in the art, including but not limited to, hook and loop fasteners, clips, or snap fasteners.

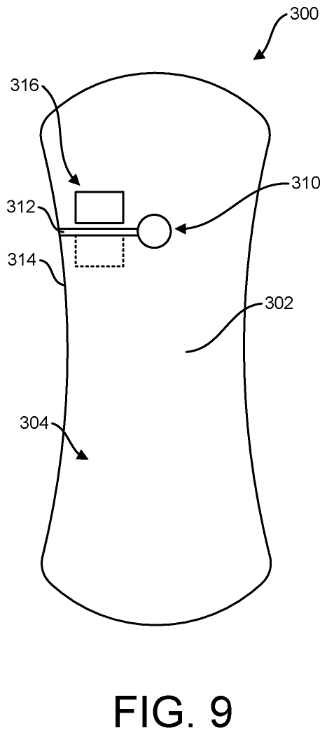

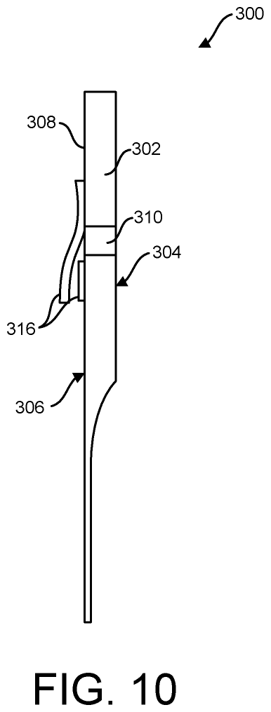

In accordance with embodiments of the invention, as illustrated in FIGS. 9-10, an absorbent pad 300 for use with a female patient wearing a catheter is provided. The absorbent pad 300 includes an absorbent material 302 operable to receive and retain fluid discharged from a catheter. The absorbent material 302 has a proximal surface 304 opposite a distal surface 306. A nonpermeable backing 308 is disposed on the distal surface 306 of the absorbent material 302 and is operable to prevent leakage of a fluid retained within the absorbent material 302. A catheter passage 310 is defined by the absorbent material 302 and the nonpermeable backing 308. The catheter passage 310 is operable to receive and form seal around the catheter 310 and is further operable to prevent the absorbent pad 300 from moving relative to the catheter. A slit 312 extends from the catheter passage 310 to an edge 314 of the absorbent pad 300 and is operable to enable a catheter 301 to be inserted into the catheter passage 310. A fastener 316 is operable to form a seal across the slit 312. The slit 312 enables the absorbent pad 300 to be attached to and removed from the catheter without removing the catheter from the patient. This is particularly advantageous as patients may wear catheters for extended periods of time and the absorbent pad 300 may become full of fluid and need to be replaced prior to the need to remove the catheter from the patient.

The male and female embodiments of the present invention are operable to function in conjunction with a catheter that is configured for a continuous flush as well as to provide patient comfort. In one embodiment, absorbent pad 300 is configured similar to most feminine hygiene pads with wider anterior and posterior diameters and a relatively more narrow middle diameter to fit the female perineal anatomy. In the embodiment illustrated in FIG. 8, the length of the absorbent pad 300 may be 18 centimeters with an anterior thickness of 1 centimeter and taper to 3 millimeters to provide more absorption near the urethral meatus.

In one embodiment, the absorbent pad 300 will have a 1 centimeter by 1 centimeter aperture in the midline of the pad at a position 5 centimeters from the anterior edge of the pad 300 operable to seat the catheter through the pad 300. The slit 312, in one embodiment, may be placed 5 centimeters from the lateral edge of the pad 300.

For the purposes of promoting and understanding of the principles of the invention, reference has been made to the preferred embodiments illustrated in the drawings, and specific language has been used to describe these embodiments. However, this specific language intends no limitation of the scope of the invention, and the invention should be construed to encompass all embodiments that would normally occur to one of ordinary skill in the art. The particular implementations shown and described herein are illustrative examples of the invention and are not intended to otherwise limit the scope of the invention in any way. For the sake of brevity, conventional aspects of the system (and components of the individual operating components of the system) may not be described in detail. Furthermore, the connecting lines, or connectors shown in the various figures presented are intended to represent exemplary functional relationships and/or physical or logical couplings between the various elements. It should be noted that many alternative or additional functional relationships, physical connections or logical connections may be present in a practical device. Moreover, no item or component is essential to the practice of the invention unless the element is specifically described as “essential” or “critical.” Numerous modifications and adaptations will be readily apparent to those skilled in this art without departing from the spirit and scope of the present invention.

1. An absorbent cuff for use with a male patient wearing a catheter, comprising:
a. a nonpermeable body, said body having a distal end opposite a proximal end, said body defining an interior operable to receive and enclose an absorbent material, said body operable to prevent leakage of a fluid retained within an absorbent material contained within the body;
b. a circumferentially concave opening disposed at the proximal end of the body, said opening operable for positioning on the catheter below the head of a patient's penis and surround the point of insertion of the catheter into the patient's urethra;
c. an absorbent material disposed within the interior of the body, said absorbent material substantially filling the interior defined by the body and the circumferentially concave opening, said absorbent material defining a circumferentially concave receiving surface disposed within the circumferentially concave opening of the body, said absorbent material defining a catheter passage operable to enclose a catheter, said absorbent material operable to receive, and retain fluid discharged from a catheter; and
d. a slit along a length of the body and absorbent material operable to enable a catheter to be inserted into and removed from the catheter passage of the absorbent material.

2. The absorbent cuff of claim 1, wherein the absorbent material is a super absorbent polymer.

3. The absorbent cuff of claim 2, wherein the absorbent material is operable to wick fluid discharged from the catheter.

4. The absorbent cuff of claim 3, wherein the absorbent material is non-abrasive and non-irritant to skin of the patient.

5. The absorbent cuff of claim 1, wherein the nonpermeable body is made of a material operable to change color to indicate the fluid level contained within the absorbent material.

6. The absorbent cuff of claim 1, wherein the absorbent device is secured circumferentially by a plurality of hook and loop fasteners.

7. An absorbent cuff for use with a male patient wearing a catheter, comprising:
a. a nonpermeable body having two opposing halves, said body having a distal end opposite a proximal end, said body defining an interior operable to receive and enclose an absorbent material, said body operable to prevent leakage of a fluid retained within an absorbent material;
b. a locking mechanism operable to secure the two opposing halves, said locking mechanism operable to create a leakproof seal along the length of the body;
c. an absorbent material disposed within the interior of the body, said absorbent material defining a catheter passage operable to enclose a catheter, said absorbent material operable to receive and retain fluid discharged from a catheter; and
d. a circumferentially concave opening disposed at the proximal end of the body, said opening operable for positioning on the catheter below the head of a patient's penis and surround the point of insertion of the catheter into the patient's urethra.

8. The absorbent cuff of claim 7, further comprising a hinge disposed along a length of each of the two opposing halves, said hinge operable to open the body, and said hinge operable to form a leakproof seal.

9. The absorbent cuff of claim 7, wherein the absorbent material is a super absorbent polymer.

10. The absorbent cuff of claim 9, wherein the absorbent material is operable to wick fluid discharged from the catheter.

11. The absorbent cuff of claim 10, wherein the absorbent material is non-abrasive and non-irritant to skin of the patient.

12. The absorbent cuff of claim 7, wherein the nonpermeable body is made of a material operable to change color to indicate the fluid level contained within the absorbent material.

13. An absorbent pad for use with a female patient wearing a catheter, comprising:
a. an absorbent material operable to receive and retain fluid discharged from a catheter, said absorbent material having a proximal surface opposite a distal surface;
b. a nonpermeable backing disposed on the distal surface of the absorbent material, said nonpermeable backing operable to prevent leakage of a fluid retained within the absorbent material;
c. a catheter passage defined by the absorbent material and the nonpermeable backing, said passage operable to receive and form seal around the catheter, said passage further operable to prevent the absorbent pad from moving relative to the catheter; and
d. a slit extending from the catheter passage to an edge of the absorbent pad, said slit operable to enable a catheter to be inserted into the catheter passage; and e. a fastener operable to form a seal across the slit.

14. The absorbent pad of claim 13, further comprising a taper at a posterior end of the pad.

15. The absorbent pad of claim 13, wherein the fastener is a hook and loop fastener.

16. The absorbent pad of claim 13, wherein the fastener is an adhesive.

17. The absorbent pad of claim 13, wherein the fastener is a plurality of snaps.

18. The absorbent pad of claim 13, wherein the absorbent material is super absorbent polymer.

19. The absorbent cuff of claim 18, wherein the absorbent material is operable to wick fluid discharged from the catheter.

20. The absorbent cuff of claim 19, wherein the nonpermeable backing is made of a material operable to change color to indicate the fluid level contained within the absorbent material.
